(19) United States
(12) Patent Application Publication
(10) Pub. No.: US 2022/0087873 A1
(43) Pub. Date: Mar. 24, 2022

(54) TWO PIECE ABSORBENT ARTICLE

(71) Applicant: The Procter & Gamble Company, Cincinnati, OH (US)

(72) Inventors: Yin Chen, Konigstein (DE); Sara Lynn Giovanni, Cincinnati, OH (US)

(21) Appl. No.: 17/480,375

(22) Filed: Sep. 21, 2021

Related U.S. Application Data
(60) Provisional application No. 63/080,864, filed on Sep. 21, 2020.

(51) Int. Cl. A61F 13/15; A61F 13/511; A61F 13/475; A61F 13/84

(52) U.S. Cl. CPC A61F 13/15203; A61F 13/84; A61F 13/4758; A61F 13/511

(57) ABSTRACT

An outer cover includes a first lateral end, a second lateral end, and a length Lc extending in a longitudinal direction between the first and second lateral ends; and a first longitudinal edge and a second longitudinal edge. The outer cover also includes an opening. The opening has a maximum longitudinal length, Lo, that is at least 50% of the length of the outer cover, Lc; and the opening is adapted to receive an absorbent insert.

### CROSS REFERENCE TO RELATED APPLICATIONS

This application claims the benefit of U.S. Provisional Application No. 63/080,864, filed Sep. 21, 2020, which is incorporated herein by reference in its entirety.

### FIELD

The present invention relates generally to the field of wearable absorbent articles having absorbent inserts and reusable outer covers, and more particularly, systems for combining the inserts into the outer covers.

### BACKGROUND

It has long been known that absorbent articles (e.g., diapers, adult incontinence articles, feminine hygiene pads) offer the benefit of receiving and containing urine and/or other bodily exudates (e.g., feces, menses, mixture of feces and urine, mixture of menses and urine, etc.). It has been proposed to manufacture two-piece absorbent articles with a reusable outer cover and a detachable absorbent insert that may be reusable or disposable. In this way, the insert may be made with materials known to provide even more superior performance while minimizing the amount of waste as the whole article need not be disposed.

Unlike fully disposable absorbent articles, two-piece articles require the caregiver/user to properly place the insert into the outer cover. In some configurations, the absorbent insert is at least partially enclosed by the outer cover during use such that the absorbent insert is placed within the outer cover as opposed to merely joined to a surface of the outer cover. In some known configurations, an outer cover is provided with a laterally-extending opening, allowing the absorbent insert to be inserted into the outer cover. However, in configurations where the opening is provided proximate to a lateral end of the outer cover, the caregiver must push his or her hand through the length of the article to ensure the absorbent insert is properly positioned. This act requires the caregiver to uncomfortably insert his or her hand into the opening or try to place the insert through the outer cover. Alternatively, the manufacturer is required to provide an opening large enough to permit positioning by hand, which results in more material in the outer cover, unsightly bulges and/or an opening that may not fully secure the insert. It has also been proposed to include a laterally-extending opening in other portions of the outer cover, i.e. away from the outer cover's lateral end. In such configurations, however, the caregiver must avoid exudates when removing the absorbent insert.

Therefore, there is a need for an article design that permits coupling an absorbent insert with an outer cover in an efficient manner, particularly in designs that enclose the insert within the outer cover. There is also a need for an article design that maintains the insert in position with minimal effort. There is a further need for a design that minimizes contact with exudates when removing the absorbent insert.

### SUMMARY

The present disclosure comprises an outer cover comprising a front lateral end, a rear lateral end, and a length Lc extending in a longitudinal direction between the front and rear lateral ends. The outer cover also comprises a first longitudinal edge and a second longitudinal edge; and an opening. The opening may be disposed a maximum distance of 20 mm or less from the first longitudinal edge. The opening may be disposed generally evenly spaced between the first longitudinal edge and the second longitudinal edge of the outer cover, in other words generally coincident with a longitudinal axis of the outer cover. The opening comprises a maximum longitudinal length, Lo, that is at least 50% of the length of the outer cover, Lc; and the opening is adapted to receive an absorbent insert.

### BRIEF DESCRIPTION OF THE DRAWINGS

FIG. 1 is a perspective view of a wearable absorbent article as it might appear being worn by a wearer about the lower torso;

FIG. 2 is a plan view of an outer cover opened and laid flat, garment-facing surface facing the viewer;

FIG. 2A is a perspective view of an outer cover with the wearer-facing surface facing the viewer;

FIG. 3 is a plan view of an absorbent insert opened and laid flat, garment-facing surface facing the viewer;

FIGS. 3A-3B are cross sectional views of exemplary absorbent inserts;

FIG. 4 is a perspective view of an absorbent article in an in-use configuration with an absorbent insert disposed inside an outer cover;

FIG. 5 is a perspective view of an outer cover;

FIG. 6 is a perspective view of an outer cover showing absorbent insert placement; and

FIG. 7 is a plan view of an absorbent article of the present disclosure.

### DETAILED DESCRIPTION

“Absorbent article” means a device that absorbs and contains body exudates and, more specifically, devices that are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, and the like.

“Absorbent insert” and “insert” mean a component of a wearable absorbent article that is adapted to contain and/or absorb urine, feces, menses or any combination thereof, and is adapted to be installable and removable as a modular unit, from an outer cover. Herein, an absorbent insert may also be referred to as an “absorbent assembly”. The terms “absorbent insert,” “insert” and “absorbent assembly” may be used interchangeably herein.

“Disposed” refers to an element being located in a particular place or position. A feature that is disposed on a surface or side of a component may be integral with said component or may be joined to said component.

“Disposable”, when referring to an absorbent insert, means that the absorbent insert is not adapted or intended to be effectively sanitarily laundered in an ordinary household laundering process and ordinary household equipment, and thereby is ordinarily unsuitable for sanitary and effective reuse so as to provide as-new intended functions and performance, following soiling by exudates and removal from an outer cover. By way of non-limiting examples, effective laundering may be frustrated or prevented, causing the insert to be disposable, by inclusion of materials and/or construction: that do not retain their substantial as-new physical shape or structure through ordinary household laundering and drying so as to be effective as-new in reuse; that absorb aqueous liquids and cannot be sufficiently dried/dehydrated in ordinary household drying equipment and ordinary drying cycles so as to be effective as-new in reuse; that dissolve or substantially degrade in ordinary household laundering or drying, causing the insert to be substantially damaged or rendered useless; and/or that cannot be effectively cleaned of exudate material through ordinary laundering, so as to be sanitary and otherwise acceptable for re-use.

“Inboard,” with respect to a first feature of an article and its position relative a second feature or location on the article, means that the first feature lies closer to a respective axis of the article than the second feature or location, along a horizontal x-y plane approximately occupied by the article when laid out flat, extended to the full longitudinal and lateral dimensions of its component web materials against any contraction induced by any included pre-strained elastomeric material, on a horizontal surface. Laterally inboard means the first feature is closer to the longitudinal axis, and longitudinally inboard means the first feature is closer to the lateral axis. Conversely, “outboard,” with respect to a first feature of an article and its position relative a second feature or location on the article, means that the first feature lies farther from the respective axis of the article than the second feature or location.

“Joined” or “attached” means configurations whereby an element is directly secured to another element by affixing the element directly to the other element, and configurations whereby an element is indirectly secured to another element by affixing the element to intermediate member(s) that in turn are affixed to the other element.

“Longitudinal” means a direction lengthwise in a component such that the longitudinal direction runs parallel to the maximum linear dimension in the x-y plane of the component. In an absorbent article as described herein, the longitudinal direction runs substantially perpendicular from a waist end edge to an opposing waist end edge when the absorbent article is in a flat out, uncontracted state, or from a waist end edge to the bottom of the crotch in a bifolded article.

“Lateral” refers to a direction generally perpendicular to the longitudinal direction. In the absorbent article described herein, the lateral direction runs substantially parallel from a side edge to an opposing side edge.

“Outer cover” means a component of a wearable absorbent article that is adapted to be worn about the lower torso of a wearer, and is adapted to support an absorbent insert and hold the insert close to the wearer's body.

“Reusable”, when referring to a component means that the component is adapted to be used for its intended purpose after initial use without substantial destruction of any portions of the component necessary for as-new functionality. For example, a reusable outer cover means an outer cover that is adapted to permit removal of at least a first insert, and replacement thereof with at least a second insert, without substantial destruction of any portions of the outer cover that are necessary to provide the substantial as-new functionality of the outer cover, and without the necessity of any repair or reconstruction following such insert replacement.

### Overview

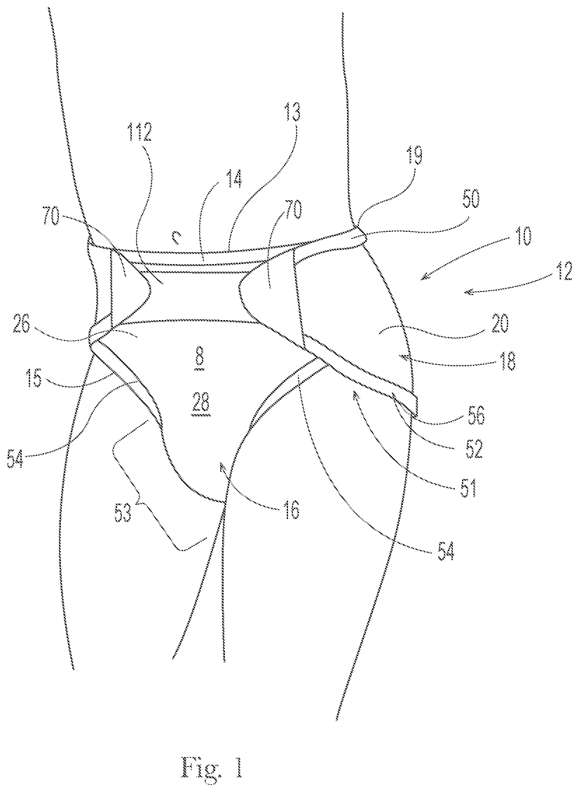

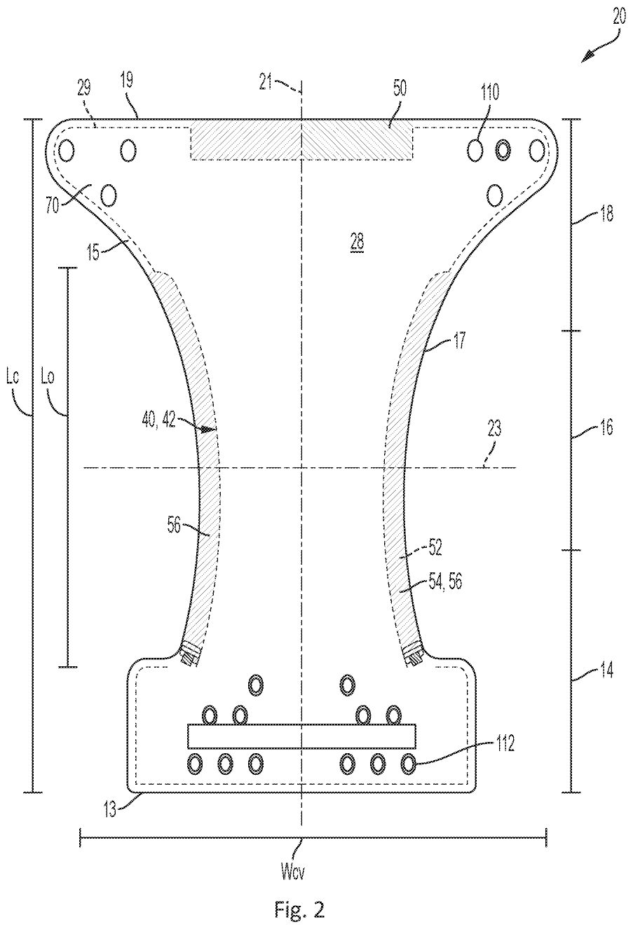

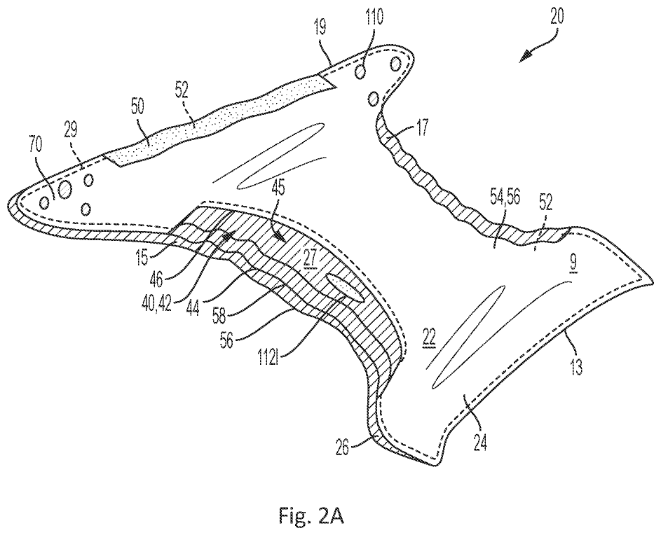

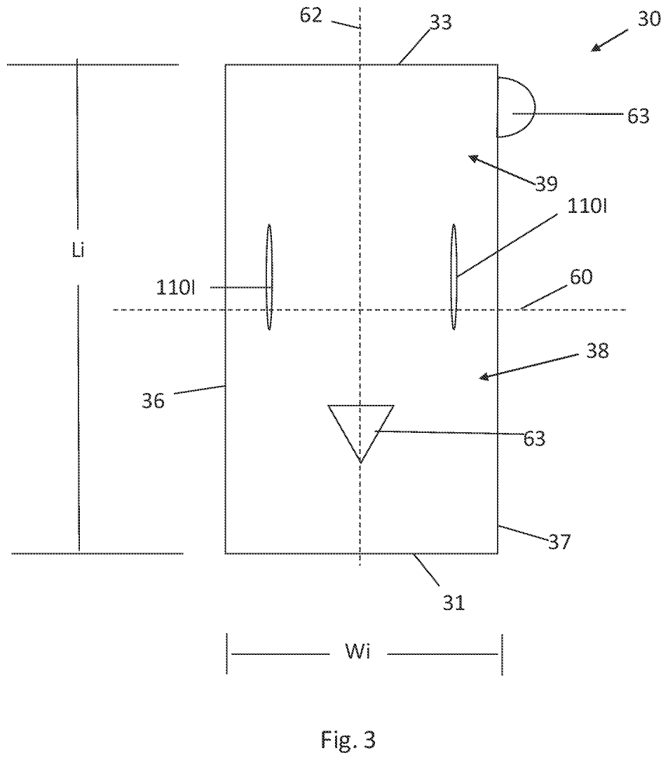

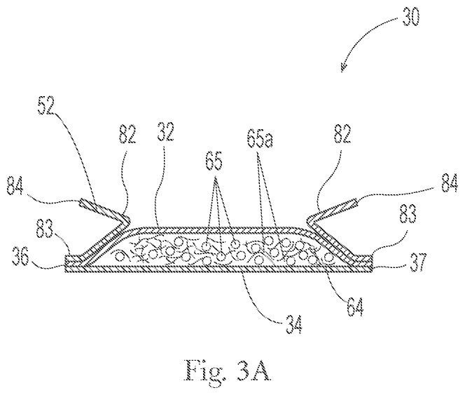

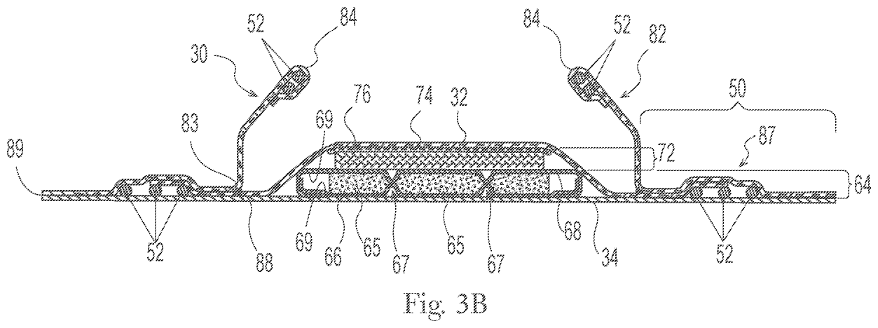

FIG. 1 is a perspective view an exemplary, nonlimiting embodiment of an absorbent article 10. The article is shown in the form of a two-piece absorbent article 12, having an outer cover 20 and an absorbent insert 30 shown in FIGS. 2-3. It is to be understood that during manufacturing, the article may comprise several discrete pieces that are joined together. However, by two-piece absorbent article, it is meant that the article in its final form has two components that the user assembles together for wear. While shown as a two-piece absorbent article, it is also contemplated that the article may comprise more than two pieces, such as attachable ears or belts. Returning to FIG. 1, it can be seen that the absorbent article 10 may have a front region 14, a rear region 18 and a crotch region 16 disposed between the front and rear regions. The article may be placed on a wearer by wrapping the outer cover 20 between the wearer's legs and under the buttocks such that the crotch region 16 is between the wearer's legs. When the insert 30 has been installed into or onto the outer cover 20, the insert 30 will then be disposed proximate to the wearer. Nonlimiting examples of two-piece articles are disclosed in U.S. Pat. Nos. 8,998,870, 9,089,456, 8,435,223, 9,011,402, 8,808,263, 8,759,605 and 8,932,273 and 9,078,789. In various embodiments, the outer cover is reusable.

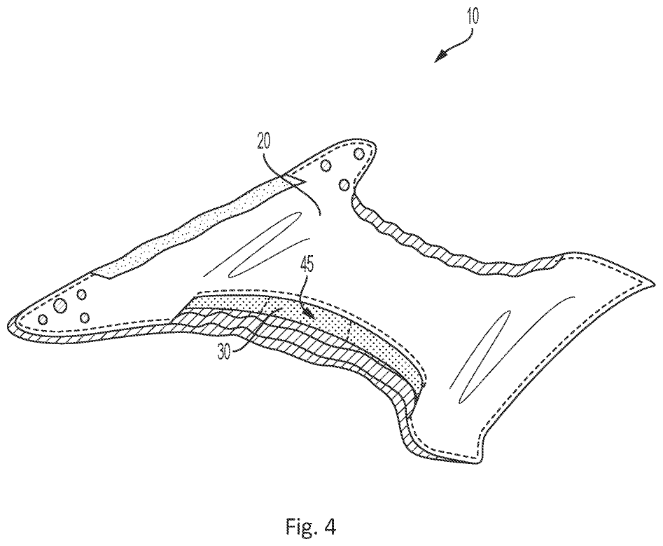

In the in-use configuration, the outer cover may at least partially enclose the absorbent insert 30 as shown in FIG. 4, forming a pocket 45 in which the absorbent insert resides during use. The insert 30 may be placed into the outer cover through one or more openings 40. As shown in FIGS. 2 and 4, at least one opening 40 is longitudinally-extending, meaning the opening extends more in the longitudinal dimension than in the lateral dimension.

### Outer Cover

FIG. 1 depicts an exemplary outer cover, with its garment-facing surface facing the viewer. FIG. 2 depicts an exemplary outer cover 20 as it may appear opened and laid substantially flat. FIG. 2A provides a schematic perspective view of an outer cover with the opening more clearly shown. In FIGS. 2-2A, the wearer-facing surface of the outer cover 20 faces the viewer. The outer cover comprises a front lateral edge 13, a rear lateral edge 19, a first longitudinal edge 15 and a second longitudinal edge 17. The outer cover comprises a length, Lc, from the outboard-most portion of the front lateral edge to the outboard-most portion of the rear lateral edge. The outer cover may comprise a lateral axis 23, dividing evenly its length, and a longitudinal axis 21. The longitudinal edges 15, 17 may be parallel to the longitudinal axis 21. However, for better fit, longitudinal edges 15, 17 may be curved or angled to produce, for example, an “hourglass” shape article when viewed in a plan view as shown in FIG. 2, for example. In nonlimiting examples, the outer cover may be asymmetric, for instance having a width in its rear region that is greater than the width in its front region. Thus, the width of the outer cover, Wcv, as measured between the longitudinal edges along a line parallel to the lateral axis, may vary along the longitudinal length of the outer cover. Nonlimiting examples of outer covers are disclosed in U.S. Pat. Nos. 9,387,138 and 8,435,223.

The outer cover, and article, comprises a front region 14, a rear region 18 and a crotch region 16 between the first and second waist regions.

Outer cover 20 and/or layers or portions thereof may be made of any durable or semi-durable knitted, woven or nonwoven textile or textile-like material that is appropriately compatible with skin of the intended wearer(s). Outer cover 20 may be formed of a single layer of a durable or semi-durable material, or may have two or more layers in the front region 14 and/or rear region 18. Suitable examples are described in U.S. applications Ser. Nos. 12/687,493; 12/687,412; 12/687,528; and 12/687,425 (all by Roe et al.). Non-limiting examples of fibers, nonwovens and laminates of nonwovens and films that might be considered for use as semi-durable outer cover materials may be found in U.S. Pat. Nos. 7,223,818; 7,211,531; 7,060,149; 6,964,720; 6,905,987; 6,890,872; 6,884,494; 6,878,647; and 5,518,801; and U.S. Published Applications Nos. 2008/0319407; 2008/0045917; 2007/0293111; 2007/0287983; 2007/0287348; 2007/0249254; 2007/0203301; and 2005/0164587.

The outer cover further comprises a first exterior surface 22 and a second exterior surface 28, which opposes the first exterior surface. One exterior surface will serve as the wearer-facing surface 9, and the other will serve as the garment-facing surface 8. In some embodiments, the exterior surfaces may be formed from the same materials. However, due to different functional needs of the garment-facing surface and the wearer-facing surfaces, it may be desirable that the exterior surfaces comprise different materials.

In nonlimiting examples, the wearer-facing surface of the outer cover may be formed from a first material or first combination of materials 24 (collectively referred to as “first material” hereinafter). The material selected may include hydrophilic fibers, or fibers treated to be hydrophilic. This may be desired in some circumstances to cause the material forming wearer-facing surface 9 to more readily absorb liquid, or transmit liquid therethrough. This may serve to provide supplemental absorbency within the outer cover for an event in which liquid exudates escape the insert, reducing the likelihood that the outer cover will leak. Alternatively, it may provide one way of communicating to the user that liquid exudates have escaped the insert, by causing wetness to be transmitted through to the outer cover such that wetness is sensed by the wearer and/or visible on exterior surfaces. Alternatively, it may serve to provide a layer that tends to draw moisture away from the skin, for a drier, more comfortable feel. Additionally, or alternatively, it may be desirable to form the wearer-facing surface from an apertured material to facilitate transfer of exudates through the wear-facing surface and onto the absorbent insert. In some nonlimiting examples, the material may include fibers having hydrophobic properties, providing enhanced liquid containment attributes to the wearer-facing layer.

The garment-facing surface of the outer cover may be formed by a second material 24, or a second combination of materials 26 (collectively referred to as “second material” hereinafter). The material selected may include fibers having hydrophobic properties, providing enhanced liquid containment attributes to such layer or may include a liquid impermeable material. In another example, however, it may be desirable in some circumstances for the selected material to include hydrophilic fibers, or fibers treated to be hydrophilic, so as will cause the material to more readily absorb liquid. This may serve to provide supplemental absorbency within the outer cover for the event in which liquid exudates escape the insert, or to provide one way of communicating to the user that liquid exudates have escaped the insert. Additionally, in some circumstances, it may be desirable that the material selected have soft tactile properties so as to have a pleasant feel that the user and/or wearer find attractive. The material also may be selected so as to have a desired appearance, including but not limited to coloration, sheen, and/or texture.

The first material 24 and the second material 26 may differ by one of the group consisting of: of texture, caliper, elasticity, opacity, water resistance, absorbency, breathability and combinations thereof.

In nonlimiting examples, the outer cover may be reversible such that both the first exterior surface and the second exterior surface are adapted to function as the wearer-facing surface and the garment-facing surface. This may provide, for example, variation in visible graphics or colors, textures and/or breathability against skin, liquid wicking properties and the like for the wearer.

Layers or other elements of the outer cover may be joined to each other via any suitable mechanism, including, for example, adhesives, mechanical bonding, ultrasonic bonding, sewing, stitching, serging, edging, and the like. In nonlimiting examples, the layers forming the first and second exterior surfaces may be joined together by a seam 29 as illustrated in FIGS. 2 and 2A, which may join the materials proximate to the first and second lateral edges and/or about the first and second longitudinal edges. The seam may be continuous. The seam may be continuous except where the opening(s) 40 exist, except where leg bands 56 are present, and/or except where a waist feature 50 exists. In nonlimiting examples, the seam may be used to form a portion of the leg band 56 and/or to form a portion of a waist feature. In some nonlimiting examples, the seam may be inboard the edge of one or both materials as shown in FIG. 2. In such examples, a second seam may be present along the edge to further secure the materials together. The seam may be formed through any suitable means, including adhesive, pressure bonding, heat bonding, stitches and combinations thereof.

### Waist Features, Leg Gasketing Systems, Ears

Referring again to FIGS. 1-2A, the article and outer cover may comprise one or more waist features 50. The waist feature may be elastic and may provide better fit about the waist of the wearer. Elasticized waist features include waistbands, waist cuffs having pockets formed from a portion of the waist feature that is unattached from the remaining portions of the outer cover, and waist panels and/or belts designed to fit securely about the abdomen of the wearer. Nonlimiting examples of elasticized waist features are disclosed in U.S. patent application Ser. Nos. 14/533,472; 15/074,675 and 62/855,001. Elasticized waist features may comprise one or more nonwoven or textile layers, which may be layers of the outer cover or discrete portions, and one or more elastic elements 52. In nonlimiting examples, the elasticized waist feature comprises elastic strands or ribbons joined to the nonwoven and/or textile layer(s). In further nonlimiting examples, the elasticized waist feature comprises a laminate of one or more textile and/or nonwoven layers and one or more elastomeric films. The elasticized waist element may comprise one or more rugosities if the elastic material is strained prior to lamination. In other nonlimiting examples, the layers of the elastic laminate may be joined at zero applied strain and subsequently activated.

In alternative embodiments, the waist feature may be inelastic. In such configurations, the waist feature may provide additional anchoring about the waist of the wearer.

Additionally, or alternatively, the article may comprise a leg gasketing system 54, portions of which may be formed by the outer cover and/or the absorbent insert. The outer cover may include one or more elastic elements 52, such as films or elastic strands, extending through all or a portion of the leg opening 51 to form a leg band portion 56. The elastic elements may be laminated with one or more nonwoven layers and/or one or more textile layers. The elastic elements may be laminated between the first and second materials 24, 26 described above. As described with respect to the waist feature, the leg band portion may include rugosities as result of the lamination process. In other nonlimiting examples, the leg band portion may be a zero strain, activated laminate. In further nonlimiting examples, materials (e.g., materials 24 and/or 26) are joined around the elastic element, at least partially enclosing the elastic element, but are not joined to the elastic elements. In some embodiments, elastic elements are affixed within the outer cover only at or near one or both of their respective ends, e.g., within a pouch, tube or envelope structure formed of outer cover material—referred to herein as a “drawstring elastic”.

The waist features and/or leg band portions may be disposed along the edge of the outer cover, and in some circumstances, it may be desired to have elasticized waist and leg band portions situated along substantially the entire length of the leg and/or waist openings so as substantially or completely encircle the wearer's legs and/or waist while outer cover 20 is worn. The gathered material within rugosities can serve to accommodate stretching of waist feature and leg band portions. This arrangement not only may provide for better fit about the wearer's legs, but also may enable the outer cover 20, when formed of appropriately sized and shaped material, to form a pouch-like structure 53 in the crotch region (see FIG. 1) when worn, which may serve to provide space within the outer cover to accommodate the insert 30 and help hold it in place within outer cover 20, in a substantially laterally centered position within the crotch region. Alternatively, or additionally, the elastic strands or strips in waist features 50 and leg band portions 56 may be affixed within the outer cover only at or near their respective ends, e.g., within a pouch, tube or envelope structure formed of outer cover material—referred to herein as a “drawstring elastic”. This will allow the elastic material and associated outer cover material to stretch and move freely and independently of each other, which may promote fit and comfort. A snug fit about the wearer's legs provided by such elasticized leg band portions 52 may serve to enhance containment capability of the wearable absorbent article. The outer cover 20 may also include anchoring supplements, bands or systems thereof as described in more detail in U.S. Pat. No. 8,932,273.

The outer cover may include ears 70 in one or both of the front and rear regions. As shown in FIGS. 1-2A, the ears may include fastening components 110 such that the ears can be secured to the opposing region (e.g., rear ears may include fastening components that may engage with the front region). The outer cover may include receiving components 112 to operatively engage with fastening components 110. Nonlimiting examples of engageable fastening and receiving components include tape tabs, hook and loop fastening components, interlocking fasteners such as tabs & slots, buckles, buttons, snaps, and/or hermaphroditic fastening components. Some exemplary surface fastening systems are disclosed in U.S. Pat. Nos. 3,848,594; 4,662,875; 4,846,815; 4,894,060; 4,946,527; 5,151,092; and 5,221,274. An exemplary interlocking fastening system is disclosed in U.S. Pat. No. 6,432,098. A fastening component may be discrete from and joined to the outer cover or may be integral with the outer cover. The receiving component may be discrete from and joined to the outer cover or may be integral with the outer cover. The fastening component and/or the receiving component may further include a release tape or other material, including folded material, that protects the component from insult prior to use. In nonlimiting examples, a fastening component 110 is disposed on a wearer-facing surface 9 of the outer cover and a receiving component 112 is disposed on a garment-facing surface 8. In such nonlimiting examples, the outer cover may comprise two or more fastening systems having distinct fastening locations, which deconcentrates lateral tensile focuses and reduces the tendency of the front portion of the article to pivot around the single fastening location. Suitable dual fastening systems are disclosed for example in U.S. patent application Ser. Nos. 16/684,230 and 62/975,836.

The outer cover may also comprise one or more insert components of an insert-to-outer cover fastening system. The outer cover may comprise fastening components capable of operatively engaging with an insert receiving component disposed on the absorbent insert, and/or may comprise receiving component(s) 1121 capable of operatively engaging with an insert fastening component 110I disposed on the absorbent insert as shown in FIGS. 2A-3. The insert fastening and receiving components may comprise any of the exemplary components disclosed. The insert fastening and/or receiving components may be disposed on an interior surface 27 of the outer cover within a pocket 45. The fastening and/or receiving components may be discrete from and joined to the outer cover or absorbent insert or may be integral with one or both as is disclosed in U.S. Pat. App. No. 62/975,836.

### Outer Cover Opening(s)

As noted above, in the in-use configuration of the present invention, the outer cover at least partially encloses the absorbent insert 30 as shown in FIG. 4, forming a pocket 45 in which the absorbent insert resides during use. The wearer-facing surface 22 of the outer cover may cover at least about 60%, or at least about 75% or at least about 80%, or at least about 90%, or from about 60% to about 100% of the surface area of the absorbent insert, when the absorbent insert is fully positioned within the pocket as designed, reciting for said range every 5% increment therein. In some nonlimiting examples, the width of the outer cover wearer-facing surface is greater than the width of the absorbent insert throughout a majority of, or all of, the length of the absorbent insert. Additionally, or alternatively, the garment-facing surface 28 of the outer cover may cover at least about 60%, or at least about 75% or at least about 80%, or at least about 90%, or from about 60% to about 100% of the surface area of the absorbent insert, when the absorbent insert is fully positioned within the pocket as designed, reciting for said range every 5% increment therein. In some nonlimiting examples, the width of the outer cover garment-facing surface is greater than the width of the absorbent insert throughout a majority of, or all of, the length of the absorbent insert.

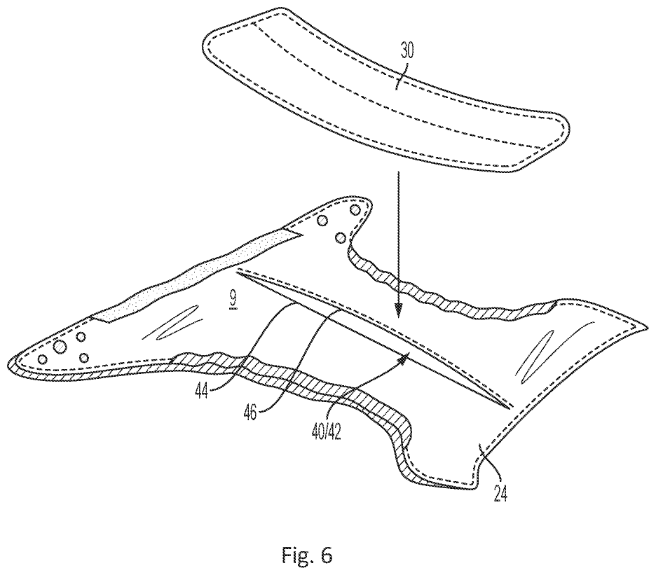

The outer cover comprises one or more openings 40 which are capable of receiving an absorbent insert 30, as shown in FIGS. 4 and 6. The openings are sized and shaped to permit the absorbent insert 30 to be inserted into the pocket 45 by the user. In various embodiments, an opening comprises a maximum longitudinal length, Lo, that is at least about 50%, or at least about 60%, or at least about 70%, or from about 50% to about 100%, or from about 55% to about 85%, or from about 60% to about 75% of the length of the outer cover, Lc, reciting for each range every 5% increment therein.

The one or more openings includes at least one longitudinally-extending opening 42. By longitudinally-extending, it is meant that the opening extends more in the longitudinal dimension than in the lateral dimension. For purposes of longitudinally-extending, the longitudinal direction includes directions within 40 degrees of the maximum linear dimension in the x-y plane of the outer cover.

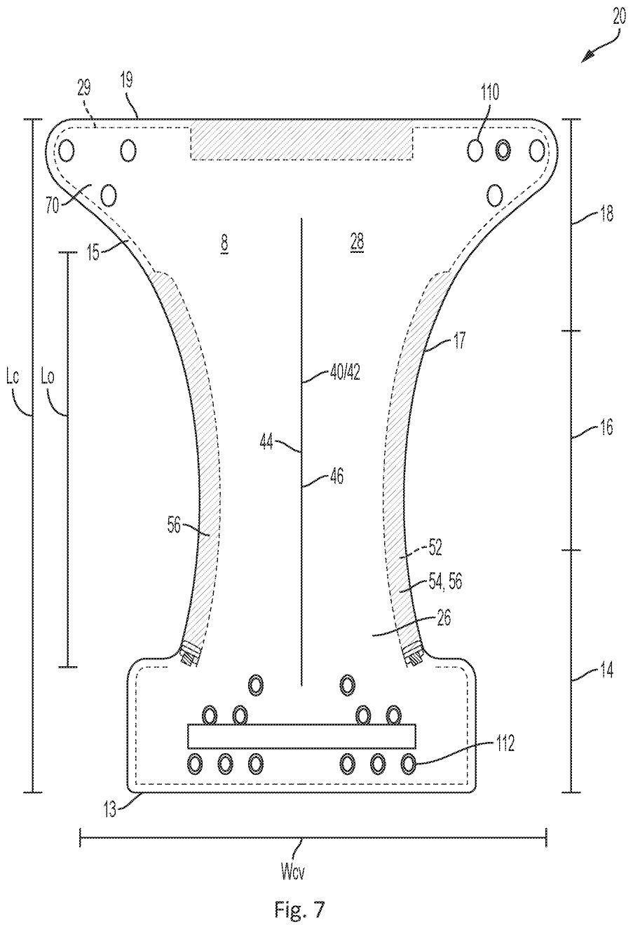

The longitudinally-extending opening 42 may be present in the crotch region 16 as shown in FIG. 2. The longitudinally-extending opening 42 may extend into one or both of the front and rear regions as shown in FIGS. 6 and 7.

Turning to FIG. 2A, the opening may comprise a first edge 44 which generally remains in the x-y plane of the outer cover when the opening is in an opened state, and a second edge 46 which extends above the x-y plane in the opened state. In various embodiments, a first edge 44 is disposed a maximum distance of 20 mm or less, or 15 mm or less, or about 10 mm or less, or from about 0 mm to about 20 mm, from the first or second longitudinal edge, reciting for said range every 1 mm increment therein. The first edge may be coincident, or at least partially coincident, with the first or second longitudinal edge.

In embodiments having a leg band 56, the first edge 44 may be disposed a maximum distance of about 15 mm or less, or about 10 mm or less, or about 5 mm or less, or from about 2 mm to about 15 mm, or from about 5 mm to about 10 mm, from an inboard edge 58 of the leg band, reciting for each range every 0.5 mm increment therein. The inboard edge 58 of the leg band may be defined by a bond or series of bonds that enclose the elastic material 52. The first edge 44 may be coincident with the inboard edge 58.

As shown in FIGS. 6 and 7, the opening 40 and/or longitudinally extending opening 42 may be disposed generally evenly spaced between the first longitudinal edge 15 and the second longitudinal edge 17 of the outer cover 20, in other words generally coincident with the longitudinal axis 21 of the outer cover 20. As shown in FIG. 6, the opening 40 may be disposed in the wearer-facing surface 9 of the outer cover 20. As shown in FIG. 7, the opening may be disposed in the garment-facing surface 8 of the outer cover 20. Disposition of the opening generally evenly spaced between the first longitudinal edge and the second longitudinal edge may make insertion of an absorbent easier, as the leading edge of the absorbent insert does not need to traverse the entire width of the outer cover in order to be properly placed.

Regardless of the placement of the opening 40 on the outer cover 20, an opening 40 may be provided on the wearer-facing surface 9 and/or on the garment-facing surface 8. In embodiments where the outer cover is formed from multiple materials 24, 26 as described above, the opening may be disposed in the first material and/or the second material. In nonlimiting examples, one or more openings are formed in the first material 24 and the second material 26 is devoid of openings. In other nonlimiting examples, one or more openings are formed in the second material 26 and the first material 24 is void of openings. As shown in FIG. 6, an opening 40 is disposed in the wearer-facing surface 9, while the garment-facing surface 8 is devoid of openings. In the arrangement shown in FIG. 6, the first edge 44 and the second edge 46 are formed from the first material 24. As shown in FIG. 7, an opening 40 is disposed in the garment-facing surface 8, while the wearer-facing surface 9 is devoid of openings. In the arrangement shown in FIG. 7, the first edge 44 and the second edge 46 are formed from the second material 26.

The outer cover 20 of the present disclosure may comprise a wearer-facing surface 9 formed from a first combination of materials 24. Additionally, or instead, the outer cover 20 may comprise a garment-facing surface 8 formed from a second combination of materials 26. A portion of a first material of the first and/or second combination of materials 24/26 may form the first edge 44 of the opening 40. A portion of a second material of the first and/or second combination of materials 24/26 may form the second edge 46 of the opening 40. At least a portion of the first material of the first combination of materials 24 may at least partially overlap at least a portion of the second material of the first combination of materials 24. Likewise, at least a portion of the first material of the second combination of materials 26 may at least partially overlap at least a portion of the second material of the second combination of materials 26. At least a portion of this overlap may form the opening 40.

The opening 40 may be sized and shaped such that the absorbent insert 30 will remain in the pocket once inserted. For example, the opening may be smaller in one or more dimensions than the insert. Additionally, or alternatively, the second edge 46 may be spaced from the first edge 44 such that less than 10 mm of void space in the z-direction exists above or below the absorbent insert within the pocket.

The opening may be sealable, such that it may be closed or repeatedly opened and reclosed. In nonlimiting examples, the first edge 44 and/or the second edge 46 may include means for attaching to each other, including any of the fastening components and/or receiving components described above, as well as pressure sensitive adhesive. Additionally, or alternatively, the first 44 or second edge 46 may be capable of wrapping about the longitudinal edge and being secured to an exterior surface.

Additionally, or alternatively, opening may be pre-sealed, such that it is opened prior to initial use. One benefit of this construction is that the user may determine whether he or she wants to utilize the opening, particularly in embodiments where the insert may be inserted through other openings or may be disposed on the wearer-facing surface of the article. Another benefit is that the interior of the outer cover may be protected from dust, moisture or other environmental effects prior to use. The pre-seal may be created in the same means described above in relation to a sealable opening. Additionally, or alternatively, the pre-seal may be made by stitching.

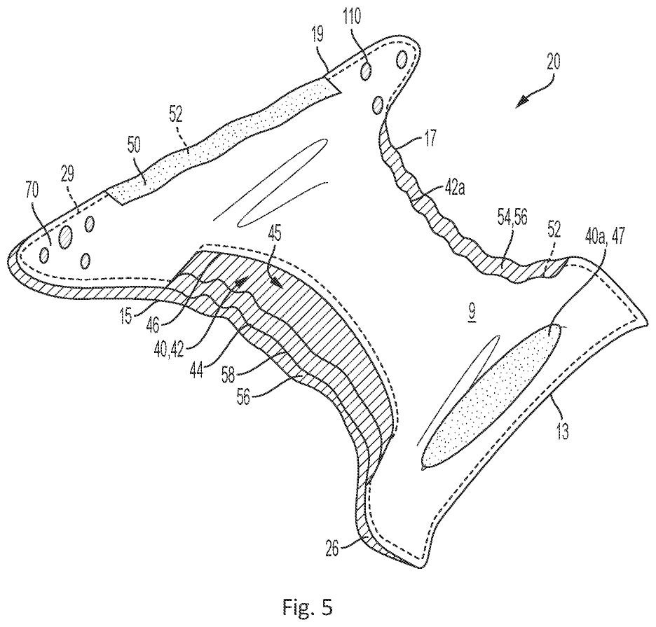

Turning to FIG. 5, the outer cover may comprise a second opening 40a (or any additional number of openings) having any of the features described above. Additional openings 40a allow for further adjustment of the insert, while minimizing the distance the user must place his or her hand within the pocket. In embodiments with multiple openings, one opening may be provided in the wearer-facing surface while another may be provided in the garment-facing surface. Any suitable combination is within the scope of the present disclosure. Additionally, or alternatively, in embodiments with multiple openings, two or more openings may differ by one of the group consisting of: shape, size (including length, width and area), orientation, material from which the opening cut/formed and combinations thereof.

In some nonlimiting examples, the outer cover may include a second longitudinally-extending opening 42a. The second longitudinally-extending opening may have any of the features discussed above with respect to the longitudinally-extending opening. By way of nonlimiting example, the second longitudinally-extending opening may have a maximum longitudinal length that is at least 50% of the length of the outer cover. The second longitudinally-extending opening may be positioned proximate to an opposing longitudinal edge (i.e., if the first opening 42 is proximate to the first longitudinal edge 15, then the second opening 42a may be proximate to the second longitudinal edge). Additionally, or alternatively, the outer cover may comprise a laterally-extending opening 47. The laterally-extending may have a lateral dimension that is greater than its longitudinal dimension. A laterally-extending opening may be disposed proximate to a waist edge 13, 19, as shown in FIG. 5. A laterally-extending opening may be disposed proximate to the lateral axis of the outer cover to shorten the distance the user's hands must travel when positioning the absorbent insert in the front and rear regions. For purposes of laterally-extending, the lateral direction includes directions within 40 degrees of the direction perpendicular to the maximum linear dimension in the x-y plane of the outer cover.

### Absorbent Insert

Returning to FIG. 3, the absorbent insert 30 may be designed to contain and/or absorb body exudates, and may be made of pliable materials as will be described further below. The insert 30 includes a forward region 38 and a rearward region 39, a first lateral end 31 and a second lateral end 33, a first longitudinal edge 36, a second longitudinal edge 37, a lateral axis 60 and a longitudinal axis 62. The insert 30 comprises a length L from the outboard-most portion of front edge 31 to the outboard-most portion of the rear edge 33. The lateral axis equally divides the length Li. In nonlimiting examples, the insert length L is less than the length of the outer cover Lc.

The longitudinal edges 36, 37 may be generally parallel to the longitudinal axis. Alternatively, the longitudinal edges 36, 37 may be curved, such as in an hour-glass configuration. Thus, the width of the insert, Wi, may vary. In nonlimiting examples, the width of the insert, Wi, is less than the width of the outer cover, Wcv, at one or more longitudinal positions. The width of the insert, Wi, may be less than the width of the outer cover, Wcv, throughout the length of the insert.

The insert also may include one or more grasp structures 63. The grasp structures may be provided to enable the user to quickly and easily grasp the insert, handle the insert during application and/or properly place the insert. For instance, in embodiments with more than one opening, the grasp structure(s) may be used to more easily pull or otherwise position the absorbent insert through or using an opening after inserting it through another opening. In certain embodiments, the grasp structure may have a different tactile feel than surrounding or adjacent areas to distinguish the area and ease the user's identification of the grasp structures.

The absorbent insert may be made of washable material such as cloth, or of a material suitable for composting, such as cellulose. In other embodiments, the absorbent insert may be disposable an made from materials commonly found in disposable absorbent articles.

As shown in FIGS. 3-3B, the insert 30 may have a topsheet 32 and a backsheet 34. The topsheet and backsheet may be joined together. An absorbent core 64 may be disposed between the topsheet and the backsheet as shown for example in FIG. 3A. As shown in FIGS. 3A and 3B, the insert 30 may further include cuffs 80 affixed along the longitudinal sides 36, 37. Nonlimiting examples of absorbent inserts and details of their features are disclosed in U.S. Pat. Nos. 8,546,64 and 9,011,402. Such cuffs may be capable of extending through one or more openings 40.

### Topsheet

The topsheet 32 is generally a portion of the insert 30 that may be positioned at least in partial contact or close proximity to a wearer. Suitable topsheets 32 may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. The topsheet 32 is generally supple, soft feeling, and non-irritating to a wearer's skin. Generally, at least a portion of the topsheet 32 is liquid pervious, permitting liquid to readily penetrate through the thickness of the topsheet 32. The topsheet 32 may comprise one or more apertures.

Any portion of the topsheet 32 may be coated with a lotion, antibacterial or skin care composition as is known in the art. The topsheet 32 may be fully or partially elasticized or may be foreshortened so as to provide a void space between the topsheet 32 and the core 64.

Topsheet 32, backsheet 34 or any portion of the topsheet or backsheet may be embossed and/or matte finished to provide a more cloth-like appearance.

### Backsheet

Backsheet 34 is generally the outer liner portion of insert 30 forming the garment-facing surface 64 thereof, and prevents the exudates absorbed and contained within insert 30 from wicking through and soiling the outer cover. The backsheet 34 may comprise one or more nonwovens, films, elastomeric films, foams, strands, or combinations of these or other suitable materials with nonwovens or synthetic films. In nonlimiting examples, the backsheet is a laminate of an elastomeric material, such as a film, and a nonwoven.

In various embodiments, the backsheet 34 is substantially water-impermeable. Suitable backsheet 34 materials include nonwovens and/or films. Other suitable backsheet 34 materials may include breathable materials that permit vapors to escape from the insert 30 while still preventing exudates from passing through the backsheet 34. Exemplary breathable materials may include materials such as woven webs, nonwoven webs, composite materials such as film-coated nonwoven webs, and microporous films.

In certain embodiments, the backsheet 34 may have a water vapor transmission rate (WVTR) of greater than about 2000 g/24 h/m², greater than about 3000 g/24 h/m², greater than about 5000 g/24 h/m², greater than about 6000 g/24 h/m², greater than about 7000 g/24 h/m², greater than about 8000 g/24 h/m², greater than about 9000 g/24 h/m², greater than about 10000 g/24 h/m², greater than about 11000 g/24 h/m², greater than about 12000 g/24 h/m², greater than about 15000 g/24 h/m², measured according to WSP 70.5 (08) at 37.8° C. and 60% Relative Humidity. A higher WVTR may be desired in this particular application, since the insert backsheet 34 will not form the outer surface of the wearable article, as a conventional disposable diaper backsheet would, but rather, will be covered by the one or more layers of the outer cover material(s)—which themselves may act in some circumstances to reduce WVTR of the composite structure.

Other suitable materials and/or manufacturing techniques may be used to provide a suitable backsheet 34 including, but not limited to, surface treatments, particular film selections and processing, particular filament selections and processing.

Backsheet 34 may be joined to topsheet 32, absorbent core 64 or any other element of insert 30 by any suitable attachment mechanism known in the art. For example, the attachment mechanism may include a continuous line or layer of adhesive, a patterned layer of adhesive, or an array of separate lines, spirals, or spots of adhesive. One example of an attachment mechanism comprises an open pattern network of filaments of adhesive as disclosed in U.S. Pat. No. 4,573,986. Other suitable attachment mechanisms include several lines of adhesive filaments which are swirled into a spiral pattern, as is illustrated by the apparatus and methods shown in U.S. Pat. Nos. 3,911,173; 4,785,996; and 4,842,666. Adhesives which have been found to be satisfactory are manufactured by H. B. Fuller Company of St. Paul, Minn. and marketed as HL-1620 and HL 1358-XZP. Alternatively, the attachment mechanism may comprise heat bonds, pressure bonds, ultrasonic bonds, dynamic mechanical bonds, stitches or any other suitable attachment mechanisms or combinations of these attachment mechanisms known in the art.

It will be appreciated that the outer cover described above can be constructed of materials and construction so as to bear and sustain a majority of the structural loading generally imposed upon a disposable diaper, by stretching and accommodation of the wearer's anatomical features and body movements, and by absorption, swelling and added weight resulting from the wearer's exudations of waste. Thus, lesser requirements for structural strength of an insert might be present with use of such an outer cover, as compared with strength required of inside components of a disposable diaper. Therefore, an article such as described herein may include a disposable absorbent insert manufactured from materials that are different from those ordinarily used in the manufacture of disposable diapers, such as petroleum-derived materials, e.g., polyethylene and polypropylene. For example, a disposable absorbent insert having one or more of a topsheet, backsheet, standing cuffs and/or other components formed of products of wood, cotton, flax (linen), hemp, bamboo, or other cellulose fibers (e.g., paper), in addition to the materials identified above, is contemplated. If resistance to aqueous liquid penetration or substantial liquid impermeability is desired, e.g., for a backsheet and/or standing cuffs, a material formed of ordinarily hydrophilic fibers such as paper may be coated or impregnated with a hydrophobic material, such as a skin-compatible oil or wax, to impart the desired resistance to aqueous liquid penetration. Each of the materials forming the insert may be selected so as to be dispersible in water or an aqueous solution, flushable, biodegradable and/or compostable (preferably to an agriculturally usable humus or soil amendment).

### Absorbent Core

Turning to FIG. 3A, the insert 30 may have an absorbent core 64 disposed within the envelope-like structure formed by the topsheet 32 and backsheet 34. The absorbent core 64 may comprise materials suitable for absorbency and for washing and reuse of the insert. In nonlimiting examples, the insert may be formed of cotton, fleece, flannel, felt, nylon, polyester, terry cloth and combinations thereof.

Alternatively, the absorbent core 64 may comprise a wide variety of liquid-absorbent materials 65 commonly used in disposable diapers and other absorbent articles. Examples of suitable absorbent materials include comminuted wood pulp, which is generally referred to as air felt creped cellulose wadding; melt blown polymers, including co-form; chemically stiffened, modified or cross-linked cellulosic fibers; tissue, including tissue wraps and tissue laminates; absorbent foams; absorbent sponges; superabsorbent polymers; absorbent gelling materials (AGM); or any other known absorbent material or combinations of materials. The absorbent core may have an absorbent capacity of at least about 120 g, or at least about 150 g, or at least about 200 g, or from about 120 to about 300 g, reciting for said range every 10 increment therein, as determined by the Core Capacity Test Method herein.

In some embodiments, the core is void of superabsorbent polymers and/or absorbent gelling materials. In other embodiments, at least a portion of the absorbent core is substantially cellulose free and contains less than 10% by weight cellulosic fibers, less than 5% cellulosic fibers, less than 1% cellulosic fibers, no more than an immaterial amount of cellulosic fibers or no cellulosic fibers. It should be understood that an immaterial amount of cellulosic material does not materially affect at least one of the thinness, flexibility, and absorbency of the portion of the absorbent core that is substantially cellulose free. Among other benefits, it is believed that when at least a portion of the absorbent core is substantially cellulose free, this portion of the absorbent core is significantly thinner and more flexible than a similar absorbent core that includes more than 10% by weight of cellulosic fibers. The amount of absorbent material, such as absorbent particulate polymer material 65a present in the absorbent core may vary, but in certain embodiments, is present in the absorbent core in an amount greater than about 80%, or greater than about 85%, or greater than about 90%, or greater than about 95% by weight of the core. In nonlimiting examples, a thermoplastic material, such as a thermoplastic adhesive composition 66, may be used to immobilize superabsorbent particles on a substrate (e.g., the topsheet, backsheet or core wrap).

In some embodiments, as shown in FIG. 3B, the absorbent core may comprise one or more channels 67, wherein said channels are substantially free of absorbent particulate polymer material. The channels 67 may extend longitudinally or laterally. The absorbent core may further comprise two or more channels. The channels may be straight, curvilinear, angled or any workable combination thereof. In nonlimiting examples, two channels are symmetrically disposed about the longitudinal axis.

The absorbent core 64 may include a core wrap 68, comprising one or more substrates 69 to encloses the absorbent material 65. Where channels are present, the core wrap may be bonded within one or more channels, thereby providing permanent channels which maintain their channel structure in the wet state.

The absorbent core 64 may be manufactured in a wide variety of sizes and shapes (e.g., rectangular, hourglass, “T”-shaped, etc.). The configuration and construction of absorbent core 64 may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures).

### Acquisition-Distribution System

Referring to FIGS. 3B, in some embodiments, an acquisition-distribution system (ADS) 72 is disposed between the topsheet 32 and the absorbent core 64. One function of the ADS is to quickly acquire one or more of the fluids and distribute them to the absorbent core in an efficient manner The ADS may comprise one, two or more layers, which may form a unitary layer or may remain as discrete layers which may be attached to each other. The ADS 72 may include hydrophilic materials that provide significant wicking of bodily exudates. These materials may dewater the topsheet 32 and quickly move bodily exudates into the absorbent core 64. The ADS 72 may comprise one or more nonwoven materials, foams, cellulosic materials, cross-linked cellulosic materials, air laid cellulosic nonwoven materials, spunlace materials, or combinations thereof, for example. In some instances, portions of the ADS 72 may extend through portions of the topsheet 32, portions of the topsheet 32 may extend through portions of the ADS 72, and/or the topsheet 32 may be nested with the ADS 72. Typically, an ADS 72 may have a width and length that are smaller than the width and length of the topsheet 32. The ADS may have one or more channels as described above with reference to the absorbent core 64. The channels in the ADS may align or not align with channels 67 in the absorbent core 64. In an example, a first acquisition material 74 may comprise a nonwoven material and as second acquisition material 76 may comprise a cross-linked cellulosic material.

### Cuff Structures

As noted above, the article 10 may comprise a leg gasketing system 50, portions of which may be formed by the insert 30. Such portions may be exposed through an opening 40 in the outer cover when the absorbent insert is in the in-use configuration. Returning to FIGS. 3A-3B, the insert may comprise a pair of longitudinal standing cuffs 82, also referred to as barrier leg cuffs. Each standing leg cuff may be formed by a piece of material which is bonded to the absorbent insert so it may extend upwards from a wearer-facing surface and provide improved containment of fluids and other body exudates approximately at the junction of the torso and legs of the wearer. The standing leg cuffs are delimited by a proximal edge 83 joined directly or indirectly to the topsheet 32 and/or the backsheet 34 and a free terminal edge 84, which is intended to contact and form a seal with the wearer's skin. In some embodiments, the free terminal edge 84 comprises a folded edge. The standing leg cuffs 82 extend at least partially between the front edge 31 and the rear edge 33 on opposite sides of the longitudinal centerline 62 and are at least present in the crotch region.

The standing leg cuffs may be integral with the topsheet 32 or the backsheet 34 or may be a separate material joined to the topsheet and/or backsheet. Each standing leg cuff 82 may comprise one, two or more elastic elements 52 close to the free terminal edge 84 to provide a better seal. The standing cuff may be formed of any of a variety of substrates such as plastic films and woven or nonwoven webs of natural fibers (e.g., wood or cotton fibers), synthetic fibers (e.g., polyester or polypropylene fibers), or a combination of natural and synthetic fibers. In certain embodiments, the standing cuffs may be formed of a substantially liquid impermeable web to contain and isolate liquid exudates from the outer cover, outer clothing and environment of the wearer and/or may be formed of a vapor permeable web for breathability of the insert and article.

Referring to FIG. 3B, the article 10 may comprise a dual gasketing system, which includes the standing cuffs 82 and gasketing cuffs 87. The gasketing cuffs 87 may be joined to the insert 30, more particularly to the topsheet and/or backsheet. The gasketing cuffs are disposed outboard of the standing cuffs and may provide a better seal around the thighs of the wearer. A gasketing cuff 87 may comprises a proximal edge 88 and a free terminal edge 89. The free terminal edge 89 may comprise a folded edge. Each gasketing cuff may comprise one or more elastic elements 52, which may be sandwiched between other layers of material, such as the portions of material forming the attached proximal portions of the standing cuffs, topsheet, backsheet, separate gasketing cuff material, or combinations thereof. The gasketing cuff may be formed of any of a variety of substrates such as plastic films and woven or nonwoven webs of natural fibers (e.g., wood or cotton fibers), synthetic fibers (e.g., polyester or polypropylene fibers), or a combination of natural and synthetic fibers. As noted with respect to standing cuffs 82, gasketing cuffs may likewise be formed of any suitable web materials but preferably are formed of web materials that are effectively liquid impermeable while being vapor permeable, so as to contain the wearer's liquid exudates within the insert while permitting the insert to “breathe” to avoid excess humidity within the insert (which may overhydrate the wearer's skin and promote conditions such as diaper rash). Suitable materials include nonwoven, films, elastic strands and combinations thereof. In further embodiments, the leg gasketing system comprises standing leg cuffs that are integral with gasketing cuffs. Suitable leg gasketing systems which may be part of the absorbent article are disclosed in U.S. Pat. Nos. 8,939,957; 3, 860,003; 7,435,243; 8,062,279.

### Bio-Sourced Materials

Components of the disposable absorbent article can at least partially be comprised of bio-sourced content as described in U.S. Pat. Pub. Nos. 2007/0219521A1, 2011/0139658A1, 2011/0139657A1, 2011/0152812A1, and 2011/0139659A1. These components include, but are not limited to, topsheets, backsheet films, backsheet nonwovens, side panels, leg gasketing systems, superabsorbent, acquisition layers, core wrap materials, adhesives, outer covers, fastener systems, and landing zones. In at least one embodiment, a disposable absorbent article component comprises a bio-based content value from about 10% to about 100%, or from about 25% to about 75%, or from about 50% to about 60% using ASTM D6866-10, method B. In order to apply the methodology of ASTM D6866-10 to determine the bio-based content of any component, a representative sample of the component must be obtained for testing. In at least one embodiment, the disposable absorbent article component can be ground into particulates less than about 20 mesh using known grinding methods (e.g., WILEY® mill), and a representative sample of suitable mass taken from the randomly mixed particles.

### Aspects of the Present Disclosure

The following aspects of the disclosure are exemplary only and not intended to limit the scope of the disclosure.
A. An outer cover comprising:
   a front lateral end, a rear lateral end, and a length Lc extending in a longitudinal direction between the front and rear lateral ends;
   a first longitudinal edge and a second longitudinal edge; and
   a longitudinally-extending opening, wherein the longitudinally-extending opening has a maximum longitudinal length, Lo, that is at least 50% of the length of the outer cover, Lc;
   wherein the longitudinally-extending opening is adapted to receive an absorbent insert.
B. The outer cover of paragraph A wherein the absorbent insert is reusable.
C. The outer cover of paragraph A wherein the absorbent insert is disposable.
D. The outer cover of paragraph A wherein the longitudinally-extending opening is disposed generally evenly spaced between the first longitudinal edge and the second longitudinal edge.
E. The outer cover of paragraph A wherein the longitudinally-extending opening is disposed in a wearer-facing surface of the outer cover.
F. The outer cover of paragraph A wherein the longitudinally-extending opening is sealable and/or pre-sealed.
G. The outer cover of paragraph A further comprising a second opening, wherein the second opening is adapted to receive the absorbent insert.
H. The outer cover of paragraph G wherein the second opening is disposed a maximum distance from the second longitudinal edge of 20 mm or less, and wherein the second opening comprises a second maximum longitudinal length Lo2 that is at least 50% of the length of the outer cover, Lc.
I. The outer cover of paragraph G wherein the second opening is longitudinally-extending.
J. The outer cover of paragraph G wherein the second opening is laterally-extending.
K. The outer cover of paragraph A wherein the outer cover comprises a first exterior surface and a second opposing exterior surface, and wherein the outer cover is reversible such that the first exterior surface and the second exterior surface are each capable of serving as a garment-facing surface and a wearer-facing surface of an absorbent article.
L. An outer cover comprising:
   a central longitudinal axis;
   a first material forming a wearing-facing surface and joined to a second material, the second material forming a garment-facing surface;
   a front lateral end, a rear lateral end, and a length Lc extending in a longitudinal direction between the front and rear lateral ends;
   a first longitudinal edge and a second longitudinal edge; and
   an opening that is disposed in the first material, wherein the opening is longitudinally-extending, and wherein the opening is disposed generally coincident with the central longitudinal axis; and
   wherein the opening is adapted to receive an absorbent insert.
M. The outer cover of paragraph L wherein the opening comprises a longitudinal length, Lo, that is at least 50% of the length of the outer cover, Lc.
N. The outer cover of paragraph L wherein the first material and second material are different by one the group consisting of texture, caliper, elasticity, opacity, water resistance, absorbency and combinations thereof.
O. The outer cover of paragraph L wherein the second material is devoid of openings.
P. The outer cover of paragraph L further comprising one or more leg elastics extending between the first and second material.
Q. An outer cover comprising:
   a first material forming a wearer-facing surface joined to a second material, the second material forming a garment-facing surface;
   a front lateral end, a rear lateral end, and a length Lc extending in a longitudinal direction between the front and rear lateral ends;
   a first longitudinal edge and a second longitudinal edge; and
   an opening that is disposed in the second material, wherein the opening is longitudinally-extending, and wherein the opening is disposed generally evenly spaced between the first longitudinal edge and the second longitudinal edge; and
   wherein the opening is adapted to receive an absorbent insert.
R. The outer cover of paragraph Q wherein the opening comprises a longitudinal length, Lo, that is at least 50% of the length of the outer cover, Lc.
S. The outer cover of paragraph Q wherein the first material and second material are different by one the group consisting of texture, caliper, elasticity, opacity, water resistance, absorbency and combinations thereof.
T. The outer cover of paragraph Q wherein the first material is devoid of openings.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An outer cover comprising:
a front lateral end, a rear lateral end, and a length Lc extending in a longitudinal direction between the front and rear lateral ends;
a first longitudinal edge and a second longitudinal edge; and
an opening that is disposed a maximum distance of 20 mm or less from the first longitudinal edge, and wherein the opening comprises a maximum longitudinal length, Lo, that is at least 50% of the length of the outer cover, Lc; and
wherein the opening is adapted to receive an absorbent insert.

2. The outer cover of claim 1 wherein the absorbent insert is reusable.

3. The outer cover of claim 1 wherein the absorbent insert is disposable.

4. The outer cover of claim 1 wherein the opening is coincident with the first longitudinal edge.

5. The outer cover of claim 1 wherein the opening is disposed a maximum distance of 15 mm or less from an inboard edge of a leg band.

6. The outer cover of claim 1 wherein the opening is sealable and/or pre-sealed.

7. The outer cover of claim 1 further comprising a second opening, wherein the second opening is adapted to receive the absorbent insert.

8. The outer cover of claim 7 wherein the second opening is disposed a second maximum distance from the second longitudinal edge of 20 mm or less, and wherein the second opening comprises a second maximum longitudinal length Lo2 that is at least 50% of the length of the outer cover, Lc.

9. The outer cover of claim 7 wherein the second opening is longitudinally-extending.

10. The outer cover of claim 7 wherein the second opening is laterally-extending.

11. The outer cover of claim 1 wherein the outer cover comprises a first exterior surface and a second opposing exterior surface, and wherein the outer cover is reversible such that the first exterior surface and the second exterior surface are each capable of serving as a garment-facing surface and a wearer-facing surface of an absorbent article.

12. An outer cover comprising:
a first material forming a wearing-facing surface and joined to a second material, the second material forming a garment-facing surface;
a front lateral end, a rear lateral end, and a length Lc extending in a longitudinal direction between the front and rear lateral ends;
a first longitudinal edge and a second longitudinal edge; and
an opening that is disposed in the first material, and wherein the opening is longitudinally-extending; and
wherein the opening is adapted to receive an absorbent insert.

13. The outer cover of claim 12 wherein the opening comprises a longitudinal length, Lo, that is at least 50% of the length of the outer cover, Lc.

14. The outer cover of claim 12 wherein the first material and second material are different by one the group consisting of texture, caliper, elasticity, opacity, water resistance, absorbency and combinations thereof.

15. The outer cover of claim 12 wherein the second material is devoid of openings.

16. The outer cover of claim 12 further comprising one or more leg elastics extending between the first and second material.

17. An outer cover comprising:
a first material forming a wearer-facing surface joined to a second material, the second material forming an garment-facing surface;
a front lateral end, a rear lateral end, and a length Lc extending in a longitudinal direction between the front and rear lateral ends;
a first longitudinal edge and a second longitudinal edge; and
an opening that is disposed in the second material, and wherein the opening is longitudinally-extending; and
wherein the opening is adapted to receive an absorbent insert.

18. The outer cover of claim 17 wherein the opening comprises a longitudinal length, Lo, that is at least 50% of the length of the outer cover, Lc.

19. The outer cover of claim 17 wherein the first material and second material are different by one the group consisting of texture, caliper, elasticity, opacity, water resistance, absorbency and combinations thereof.

20. The outer cover of claim 17 wherein the first material is devoid of openings.
